(19) United States
(12) Patent Application Publication
(10) Pub. No.: US 2017/0165181 A1
(43) Pub. Date: Jun. 15, 2017

(54) AGENT AND METHOD FOR THE TEMPORARY SHAPING OF KERATIN FIBERS

(71) Applicant: Henkel AG & Co. KGaA, Duesseldorf (DE)

(72) Inventors: Thorsten Knappe, Schenefeld (DE); Pamela Kaftan, Hamburg (DE); Maria Catalina Bermudez Agudelo, Hamburg (DE); Tim Bethge, Hamburg (DE)

(73) Assignee: Henkel AG & Co. KGaA, Duesseldorf (DE)

(21) Appl. No.: 15/363,382

(22) Filed: Nov. 29, 2016

(30) Foreign Application Priority Data
Dec. 15, 2015 (DE) 10 2015 225 211.6

(51) Int. Cl. A61K 8/81; A61K 8/34; B65D 83/14; A61K 8/33; A61K 8/04; A61Q 5/06; A61K 8/31

(52) U.S. Cl. CPC A61K 8/8158; A61Q 5/06; A61K 8/8152; A61K 8/34; A61K 8/31; A61K 8/315; A61K 8/33; A61K 8/046; B65D 83/752; A61K 2800/594; A61K 2800/87

(57) ABSTRACT

Cosmetic agents and methods for the temporary shaping of keratinic fibers using the cosmetic agents are provided herein. In one embodiment, the cosmetic agent for the temporary shaping of keratin fibers includes a cosmetic preparation. The cosmetic preparation contains at least one copolymer a1) which is composed at least of the following monomer units: N-tert-octylacrylamide, acrylic acid, and tert-butylaminoethyl methacrylate. The cosmetic preparation further contains at least one copolymer a2) which is composed at least of the following monomer units: (meth)acrylic acid, (meth)acrylic acid alkyl ester, and (meth)acrylic acid hydroxyalkyl ester. The proportion by weight of the copolymers a1) and a2) based on the total weight of the cosmetic preparation is about 1.0 to about 10% by weight.

### CROSS-REFERENCE TO RELATED APPLICATION

This application claims priority to German Patent Application No. 10 2015 225 211.6, filed Dec. 15, 2015, which is incorporated herein by reference in its entirety.

### TECHNICAL FIELD

This disclosure relates to a cosmetic composition for setting hair or for the temporary shaping of keratin fibers, in particular human hair, wherein the composition contains a combination of two specific copolymers.

### BACKGROUND

The temporary shaping of hairstyles for a longer duration of up to several days generally requires the application of active substances for setting purposes. Hair treatment agents which serve for temporarily shaping the hair therefore play an important role. Such temporary shaping agents usually contain synthetic polymers and/or waxes as the active setting substance. Agents for aiding the temporary shaping of keratin fibers may be prepared for example as a hairspray, hair wax, hair gel or hair mousse.

The most important property of an agent for the temporary shaping of hair, hereinafter also referred to as a styling agent, consists in giving the treated fibers the strongest possible hold in the newly created shape, that is to say a shape given to the hair. This is also called a strong hairstyle hold or a high degree of hold of the styling agent. The hold of the hairstyle is substantially determined by the type and amount of the active setting substances used, but the further constituents of the styling agent may also have an effect.

Besides a high degree of hold, styling agents must satisfy a number of further requirements. These can be roughly subdivided into properties on the hair, properties of the respective formulation, for example properties of sprayed aerosols, and properties relating to the handling of the styling agent, particular importance being assigned to the properties on the hair. In particular, mention may be made of moisture resistance, low degree of tack and a balanced conditioning effect. In addition, a styling agent should as far as possible be able to be used universally for all hair types and should be gentle on the hair and skin.

In order to meet the various requirements, a large number of synthetic polymers have already been developed as active setting substances, which are used in styling agents. These polymers can be subdivided into cationic, anionic, nonionic and amphoteric setting polymers.

Known amphoteric polymers which are used in hair setting products are based on the monomers N-tert-octylacrylamide, acrylic acid and tert-butylaminoethyl methacrylate. Such polymers and the use thereof in hair styling gels are described for example in international application WO 2011/012464 A2.

European patents EP 1719499 B1, EP 1719500 B1 and EP 1726331 B1 describe acrylate resins having the INCI name Acrylates/Hydroxy Ester Acrylates Copolymer, and the use thereof in styling agents. International patent application WO 2012/054278 A2 likewise mentions Acrylates/Hydroxy Ester Acrylates Copolymers as hair setting polymers and uses as an example Acudyne® 1000 (The Dow Chemical Company) in hair mousses.

Not every polymer and not every polymer mixture is suitable in principle for producing hair styling agents. This applies in particular to hairsprays, in which for example the viscosity and thus also the spray behavior is influenced by the polymer and the amount of polymer used.

Although suitable polymers and polymer combinations for use in the field of temporary hair shaping have been in development for some time, the results achieved to date still leave room for improvement, particularly with regard to the storage stability, the ease of application and the degree of hold of said agents. In particular, styling agents available at present are still capable of improvement since a good combination of degree of hold and long-lasting hold (high humidity curl retention) is not always sufficiently ensured.

### BRIEF SUMMARY

Cosmetic agents and methods for the temporary shaping of keratinic fibers using the cosmetic agents are provided herein. In one embodiment, the cosmetic agent for the temporary shaping of keratin fibers includes a cosmetic preparation. The cosmetic preparation contains at least one copolymer a1) which is composed at least of the following monomer units: N-tert-octylacrylamide, acrylic acid, and tert-butylaminoethyl methacrylate. The cosmetic preparation further contains at least one copolymer a2) which is composed at least of the following monomer units: (meth)acrylic acid, (meth)acrylic acid alkyl ester, and (meth)acrylic acid hydroxyalkyl ester. The proportion by weight of the copolymers a1) and a2) based on the total weight of the cosmetic preparation is about 1.0 to about 10% by weight.

In another embodiment, the method for the temporary shaping of keratin fibers includes the step of applying a cosmetic agent to the keratin fibers, the cosmetic agent includes at least one copolymer a1) which is composed at least of the following monomer units: N-tert-octylacrylamide, acrylic acid, and tert-butylaminoethyl methacrylate. The cosmetic agent further includes at least one copolymer a2) which is composed at least of the following monomer units (meth)acrylic acid, (meth)acrylic acid alkyl ester, and (meth)acrylic acid hydroxyalkyl ester. The proportion by weight of the copolymers a1) and a2) based on the total weight of the cosmetic agent is about 1.0 to about 10% by weight. The method further includes the step of temporarily fixing the keratin fibers in shape.

### DETAILED DESCRIPTION

The following detailed description is merely exemplary in nature and is not intended to limit the invention or the application and uses of the invention. Furthermore, there is not intention to be bound by any theory presented in the preceding background or the following detained description.

It is contemplated herein to provide further suitable polymer combinations which are characterized by good film-forming and/or setting properties and have a very high degree of hold without having to sacrifice flexibility and good resistance to moisture, in particular resistance to sweat and water. The polymer combinations were also intended to be suitable for producing cosmetic compositions with high chemical and physical stability, and to be easy to apply.

This has been achieved as contemplated herein by a combination of two specific, mutually different copolymers. The present disclosure provides:

1. A cosmetic agent for the temporary shaping of keratin fibers, comprising
a) a cosmetic preparation, containing
   a1) at least one copolymer which is composed at least of the following monomer units:
      N-tert-octylacrylamide,
      acrylic acid,
      tert-butylaminoethyl methacrylate;
   a2) at least one copolymer which is composed at least of the following monomer units:
      (meth)acrylic acid,
      (meth)acrylic acid alkyl ester,
      (meth)acrylic acid hydroxyalkyl ester,
wherein the proportion by weight of the copolymers a1) and a2) based on the total weight of the cosmetic preparation is about 1.0 to about 10% by weight.
   2. The cosmetic agent according to point 1, wherein the proportion by weight of the copolymers a1) and a2) based on the total weight of the cosmetic preparation is about 1.5 to about 9.0% by weight and in particular about 2.0 to about 8.0% by weight.
   3. The cosmetic agent according to any of the preceding points, wherein the at least one copolymer a1), based on its total weight, consists of the monomers N-tert-octylacrylamide, acrylic acid and tert-butylaminoethyl methacrylate in a proportion of at least 90% by weight, preferably at least 95% by weight and in particular at least 97% by weight.
   4. The cosmetic agent according to any of the preceding points, wherein the copolymer a1) consists of the monomers N-tert-octylacrylamide, acrylic acid, tert-butylaminoethyl methacrylate, methyl methacrylate and hydroxypropyl methacrylate in a proportion of at least 90% by weight, preferably at least 95% by weight and in particular at least 97% by weight.
   5. The cosmetic agent according to any of the preceding points, wherein the copolymer a1) bears the INCI name Octylacrylamide/Acrylates/Butylaminoethyl Methacrylate Copolymer.
   6. The cosmetic agent according to any of the preceding points, wherein the preparation contains, based on its total weight, about 0.1 to about 9.9% by weight, preferably about 0.5 to about 8.5% by weight and in particular about 1.0 to about 7.0% by weight copolymer a1).
   7. The cosmetic agent according to any of the preceding points, wherein the at least one copolymer a2), based on its total weight, consists of the monomers
(meth)acrylic acid
(meth)acrylic acid alkyl ester
(meth)acrylic acid hydroxyalkyl ester
   in a proportion of at least 90% by weight, preferably at least 95% by weight and in particular at least 97% by weight.
   8. The cosmetic agent according to any of the preceding points, wherein the copolymer a2) bears the INCI name Acrylates/Hydroxyesters Acrylates Copolymer.
   9. The cosmetic agent according to any of the preceding points, wherein the preparation contains, based on its total weight, about 0.1 to about 9.9% by weight, preferably about 0.5 to about 8.5% by weight and in particular about 1.0 to about 7.0% by weight copolymer a2).
   10. The cosmetic agent according to any of the preceding points, wherein the weight ratio of copolymer a1) to copolymer a2) is about 1:7 to about 7:1, preferably about 1:5 to about 5:1 and in particular about 1:3 to about 3:1.
   11. The cosmetic agent according to any of the preceding points, wherein the preparation contains, based on its total weight, about 40 to about 98% by weight, preferably about 60 to about 95% by weight and in particular about 70 to about 92% by weight polar solvent.
   12. The cosmetic agent according to any of the preceding points, wherein the preparation, based on its total weight, consists of the copolymers a1) and a2), ethanol and/or water in a proportion of at least 70% by weight, preferably at least 80% by weight and in particular at least 90% by weight.
   13. The cosmetic agent according to any of the preceding points, wherein the cosmetic preparation further comprises at least one thickener, preferably from the group of polymeric organic thickeners.
   14. The cosmetic agent according to any of the preceding points, wherein the cosmetic preparation further comprises at least one thickener from the group of anionic polymeric organic thickeners.
   15. The cosmetic agent according to any of the preceding points, wherein the cosmetic preparation further comprises at least one thickener from the group of anionic, polymeric, amphiphilic thickeners.
   16. The cosmetic agent according to any of the preceding points, wherein the cosmetic preparation further comprises at least one thickener from the group having the INCI names Acrylates/Beheneth-25 Methacrylate Copolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-20 Acrylate Copolymer, Acrylates/Palmeth-25 Acrylate Copolymer, Acrylates/Palmeth-25 Itaconate Copolymer and Acrylates/Steareth-50 Acrylate Copolymer.
   17. The cosmetic agent according to any of the preceding points, wherein the preparation contains, based on its total weight, about 0.05 to about 8.0% by weight, preferably about 0.1 to about 5.0% by weight thickener.
   18. The cosmetic agent according to any of the preceding points, wherein the agent further comprises
b) at least one propellant.
   19. The cosmetic agent according to any of the preceding points, wherein the agent further comprises
b) at least one propellant from the group consisting of propane, mixture of propane and butane, dimethyl ether and 1,1-difluoroethane.
   20. The cosmetic agent according to any of the preceding points, comprising, based on its total weight,
a) about 30 to about 70% by weight of the cosmetic preparation
b) about 30 to about 70% by weight propellant.
   21. A cosmetic product, comprising
i) a cosmetic agent according to any of points 1 to 20
ii) a dispensing device with a spray valve.
   22. The use of an agent or a product according to any of points 1 to 21 for the temporary shaping of keratin fibers, in particular human hair.
   23. A method for the temporary shaping of keratin fibers, in particular human hair, in which a cosmetic agent according to any of points 1 to 21 is applied to the keratin fibers and the keratin fibers are temporarily fixed in shape.

It has surprisingly been found in the context of the present disclosure that an improved moisture resistance of styling products can be obtained by combining two constituents which are known per se and which are already used in styling products. Other usually required properties of styling products, such as long-lasting hold, stiffness and low degree of tack are in the meantime maintained or improved. Such a good combination of properties was not to be expected even with knowledge of the individual components, and was surprising. It has been found through experimentation that a highly potentiating effect, that is to say a synergistic effect, in terms of the moisture resistance and degree of hold was obtained by combining the two components.

As contemplated herein, the term keratin fibers encompasses fur, wool and feathers, but in particular human hair.

The main constituents of the cosmetic composition as contemplated herein are the amphoteric copolymer a1) and the anionic copolymer a2), which is different from the copolymer a1).

A first main constituent of cosmetic compositions as contemplated herein is the copolymer a1). With regard to the ease of production, ease of application and cosmetic effect of cosmetic agents as contemplated herein, it has proven to be advantageous if the proportion by weight of the copolymer a1) based on the total weight of the cosmetic preparation a) is about 0.1 to about 9.9% by weight, preferably about 0.5 to about 8.5% by weight and in particular about 1.0 to about 7.0% by weight.

The copolymer a1) can be traced back to the monomers N-tert-octylacrylamide, acrylic acid, tert-butylaminoethyl methacrylate and optionally further monomers.

Preferred copolymers a1) preferably consist of the monomers N-tert-octylacrylamide, acrylic acid and tert-butylaminoethyl methacrylate in a proportion of at least 90% by weight, preferably at least 95% by weight and in particular at least 97% by weight. Preferably, the copolymers a1) are obtained by exclusively using the monomers N-tert-octylacrylamide, acrylic acid and tert-butylaminoethyl methacrylate.

Particular preference is given to copolymers a1) composed of the monomers N-tert-octylacrylamide, acrylic acid, tert-butylaminoethyl methacrylate, methyl methacrylate and hydroxypropyl methacrylate. In particular, it is particularly preferred if the copolymer a1) consists of the monomers N-tert-octylacrylamide, acrylic acid, tert-butylaminoethyl methacrylate, methyl methacrylate and hydroxypropyl methacrylate in a proportion of at least 90% by weight, preferably at least 95% by weight and in particular at least 97% by weight.

The copolymers a1) described above are sold for example under the name Amphomer® (INCI name: Octylacrylamide/Acrylates/Butylaminoethyl Methacrylate Copolymer; CAS number 70801-07-9) by the company National Starch.

The cosmetic preparations as contemplated herein contain an anionic copolymer a2) as the second main constituent.

With regard to the ease of production, ease of application and cosmetic effect of cosmetic agents as contemplated herein, it has proven to be advantageous if the proportion by weight of the copolymer a2) based on the total weight of the cosmetic preparation a) is about 0.1 to about 9.9% by weight, preferably about 0.5 to about 8.5% by weight and in particular about 1.0 to about 7.0% by weight.

The copolymer a2) can be traced back to the monomers (meth)acrylic acid, (meth)acrylic acid alkyl ester and (meth)acrylic acid hydroxyalkyl ester and optionally further monomers.

Preferred copolymers a2) preferably consist of the monomers (meth)acrylic acid, (meth)acrylic acid alkyl ester and (meth)acrylic acid hydroxyalkyl ester in a proportion of at least 90% by weight, preferably at least 95% by weight and in particular at least 97% by weight. Particularly preferred copolymers a2) are obtained exclusively from the monomers (meth)acrylic acid, (meth)acrylic acid alkyl ester and (meth)acrylic acid hydroxyalkyl ester.

The cosmetic agents of a further preferred embodiment are characterized in that the at least one copolymer a2), based on its total weight, consists of the monomers
(meth)acrylic acid
(meth)acrylic acid alkyl ester
(meth)acrylic acid hydroxyalkyl ester
   in a proportion of at least 90% by weight, preferably at least 95% by weight and in particular at least 97% by weight.

The at least one methacrylic acid may be methacrylic acid or acrylic acid.

The alkyl radical of the (meth)acrylic acid alkyl ester is preferably a C1-C8 alkyl radical, which may be linear or branched. Examples of alkyl radicals are methyl, ethyl, propyl, isopropyl, 1-butyl, 2-butyl, iso-butyl, tert-butyl, linear or branched pentyl, linear or branched hexyl, linear or branched heptyl and linear or branched octyl. More preferably, the alkyl group is a C1 to C5 alkyl group. According to one embodiment of the disclosure, two or more (meth)acrylic acid alkyl esters are contained, which differ with regard to the carbon number of the alkyl group. By way of example, a methacrylic acid C1-C3 alkyl ester and an acrylic acid C2-C5 alkyl ester are contained.

The hydroxyalkyl radical of the (meth)acrylic acid hydroxyalkyl ester may be a hydroxy-C1-C10-alkyl radical, preferably a hydroxy-C2-C5-alkyl radical. In one preferred embodiment, the (meth)acrylic acid hydroxyalkyl ester unit is a (meth)acrylic acid hydroxyethyl ester.

The proportion of the units (meth)acrylic acid, (meth)acrylic acid alkyl ester and (meth)acrylic acid hydroxyalkyl ester in the acrylate resin a2) may vary within wide limits. The proportion of the (meth)acrylic acid in the acrylate copolymer is preferably about 2 to about 50% by weight, more preferably about 5 to about 30% by weight. The proportion of the (meth)acrylic acid alkyl ester in the acrylate copolymer is preferably about 5 to about 95% by weight, more preferably about 45 to about 90% by weight. The proportion of the (meth)acrylic acid hydroxyalkyl ester in the acrylate copolymer is preferably 2 to 70% by weight, more preferably about 5 to about 30% by weight.

The weight average of the molecular weight of the anionic acrylate copolymer a2) is preferably about 130,000 to about 160,000, more preferably about 140,000 to about 150,000, determined by means of gel permeation chromatography (GPC).

The viscosity of the anionic acrylate copolymer a2) used in the cosmetic composition, with a solids content of about 44 to about 46% by weight and a pH of about 3.30 to about 4.30 at 25° C., is preferably at most 150 cPS (Brookfield LV, spindle 1, 60 rpm).

The copolymers a2) described above are sold for example under the name Acudyne® 1000 (INCI name: Acrylates/Hydroxyesters Acrylates Copolymer) by Rohm & Haas.

The proportion by weight of the copolymers a1) and a2) based on the total weight of the cosmetic preparation is 1.0 to 10% by weight. Preference is given to cosmetic preparations in which the proportion by weight of the copolymers a1) and a2) based on the total weight of the cosmetic preparation is about 1.5 to about 9.0% by weight and in particular about 2.0 to about 8.0% by weight.

Besides the total proportion by weight of the copolymers a1) and a2), the weight ratio of the copolymers a1) and a2) relative to one another also has an effect on the moisture resistance, the degree of hold and the further use properties of cosmetic agents as contemplated herein. Cosmetic agents which are particularly advantageous in technical terms are characterized in that the weight ratio of copolymer a1) to copolymer a2) is about 1:7 to about 7:1, preferably about 1:5 to about 5:1 and in particular about 1:3 to about 3:1.

Besides the copolymers a1) and copolymers a2) described above, the cosmetic preparations as contemplated herein may contain further active substances, auxiliaries and care substances.

The film-forming polymers are a first group of active substances used with preference. These film-forming polymers are not the same as the copolymer a1) or copolymer a2) described above. The proportion by weight of the film-forming polymer based on the total weight of the cosmetic preparation is preferably about 0.1 to about 8.0% by weight, preferably about 0.5 to about 6.0% by weight and in particular about 1.0 to about 4.0% by weight.

With particular preference, nonionic polymers are used as film-forming polymers. Suitable nonionic polymers are for example:
vinylpyrrolidone/vinyl ester copolymers, as sold for example under the trade name Luviskol® (BASF). Luviskol® VA 64 and Luviskol® VA 73, each vinylpyrrolidone/vinyl acetate copolymers, are preferred nonionic polymers.
cellulose ethers, such as hydroxypropylcellulose, hydroxyethylcellulose and methylhydroxypropylcellulose, as sold for example under the trade names Culminal® and Benecel® (AQUALON).
shellac.
polyvinylpyrrolidones, as sold for example under the trade name Luviskol® (BASF).
siloxanes. These siloxanes may be either water-soluble or water-insoluble. Both volatile and nonvolatile siloxanes are suitable, nonvolatile siloxanes being understood to mean those compounds having a boiling point above 200° C. at normal pressure. Preferred siloxanes are polydialkylsiloxanes, such as for example polydimethylsiloxane, polyalkylarylsiloxanes, such as for example polyphenylmethylsiloxane, ethoxylated polydialkylsiloxanes and polydialkylsiloxanes which contain amine and/or hydroxyl groups.
glycoside-substituted silicones.

Due to their cosmetic effect in combination with the copolymers a1) and a2), film-forming polymers which are used with preference as contemplated herein are in particular the polyvinylpyrrolidones (INCI name: PVP) and the vinylpyrrolidone/vinyl acetate copolymers (INCI name VP/VA Copolymer), wherein the proportion by weight of said polymers is preferably limited to amounts between about 1.0 and about 10% by weight. Particularly preferred cosmetic preparations as contemplated herein are therefore characterized in that they also contain, based on their total weight, about 1.0 to about 10% by weight polyvinylpyrrolidone and/or vinylpyrrolidone/vinyl acetate copolymer, preferably polyvinylpyrrolidone. Particularly preferred cosmetic preparations have a proportion by weight of the polyvinylpyrrolidone and/or vinylpyrrolidone/vinyl acetate copolymer of about 2.0 to about 8.5% by weight, preferably about 3.0 to about 7.0% by weight, based on the total weight of the cosmetic preparation.

To sum up, with the copolymers a1) and a2) and also the film-forming polymer a3), cosmetic agents which are particularly preferred as contemplated herein contain three mutually different polymers.

As a care substance, use may be made of protein hydrolysates and/or derivatives thereof. Protein hydrolysates are product mixtures obtained by acid-, base- or enzyme-catalyzed degradation of proteins (albumins). As contemplated herein, the term protein hydrolysates is also understood to mean total hydrolysates and also individual amino acids and derivatives thereof and also mixtures of different amino acids. The molecular weight of the protein hydrolysates which can be used as contemplated herein is between about 75, the molecular weight of glycine, and about 200,000, and preferably the molecular weight is about 75 to about 50,000 and very particularly preferably about 75 to about 20,000 daltons.

Another group of care substances is formed by vitamins, provitamins, vitamin precursors and/or derivatives thereof. As contemplated herein, preference is given to those vitamins, provitamins and vitamin precursors which are usually assigned to groups A, B, C, E, F and H.

Further care substances are glycerol, propylene glycol, panthenol, caffeine, nicotinamide and sorbitol.

As a care substance, use may be made of plant extract, but also mono- or oligosaccharides and/or lipids.

The composition of some cosmetic preparations a), in which the proportion by weight of the copolymers a1) and a2) based on the total weight of the cosmetic preparation is about 1.0 to about 10% by weight, preferably about 1.5 to about 9.0% by weight and in particular about 2.0 to about 8.0% by weight, can be found in the following tables. (Unless indicated otherwise, figures specified in % by weight relate to the total weight of the cosmetic agent.)

| | Formula 1 | Formula 2 | Formula 3 | Formula 4 | Formula 5 |
|---|---|---|---|---|---|
| Copolymer | 0.1 to 9.9 | 0.1 to 8.9 | 0.5 to 8.0 | 0.5 to 7.5 | 1.0 to 7.0 |
| a1)* | | | | | |
| Copolymer | 0.1 to 9.9 | 0.1 to 8.9 | 0.5 to 8.0 | 0.5 to 7.5 | 1.0 to 7.0 |
| a2)* | | | | | |
| Optional | ad 100 | ad 100 | ad 100 | ad 100 | ad 100 |
| additives | | | | | |

| | Formula 6 | Formula 7 | Formula 8 | Formula 9 | Formula 10 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Copolymer a2)* | 0.1 to 9.9 | 0.1 to 8.9 | 0.5 to 8.0 | 0.5 to 7.5 | 1.0 to 7.0 |
| Optional additives | ad 100 | ad 100 | ad 100 | ad 100 | ad 100 |

| | Formula 11 | Formula 12 | Formula 13 | Formula 14 | Formula 15 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer | | | | | |
| (INCI) | | | | | |
| Optional additives | ad 100 | ad 100 | ad 100 | ad 100 | ad 100 |

| | Formula 16 | Formula 17 | Formula 18 | Formula 19 | Formula 20 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer (INCI) | | | | | |
| Optional additives | ad 100 | ad 100 | ad 100 | ad 100 | ad 100 |

*according to claim 1

The cosmetic preparations as contemplated herein may be made up in various ways. The proportion by weight and the precise composition of the liquid carrier optionally contained in the cosmetic preparation has a significant influence on the final made-up form of these preparations.

Preferred cosmetic agents are based on an aqueous, aqueous/alcoholic or alcoholic carrier. For instance, preferred cosmetic agents contain, based on their total weight, 40 to 98% by weight, preferably about 60 to about 95% by weight and in particular about 70 to about 92% by weight polar solvent, preferably polar solvent from the group consisting of water, ethanol and isopropanol.

As already mentioned, the lower alcohols having 1 to 4 carbon atoms which are usually used for cosmetic purposes, such as for example ethanol and isopropanol, may in particular be contained as alcohols.

Besides these alcoholic solvents, water-soluble co-solvents can also be used, particularly in combination with water. Examples of particularly preferred co-solvents are glycerol and/or ethylene glycol and/or 1,2-propylene glycol, which are preferably used in an amount of 0 to 30% by weight based on the cosmetic preparation a).

Together with the copolymers a1) and a2) described above, the aqueous, aqueous/alcoholic or alcoholic carriers preferably form a main constituent of cosmetic preparations a) as contemplated herein. Particular preference is given to cosmetic preparations which, based on their total weight, consist of the copolymers a1) and a2), ethanol and/or water in a proportion of at least 70% by weight, preferably at least 80% by weight and in particular at least 90% by weight.

The composition of some technically advantageous cosmetic preparations a) having a liquid carrier, in which the proportion by weight of the copolymers a1) and a2) based on the total weight of the cosmetic preparation is about 1.0 to about 10% by weight, preferably about 1.5 to about 9.0% by weight and in particular about 2.0 to about 8.0% by weight, can be found in the following tables. (Unless indicated otherwise, figures specified in % by weight relate to the total weight of the cosmetic agent.)

| | Formula 21 | Formula 22 | Formula 23 | Formula 24 | Formula 25 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |

| | Formula 26 | Formula 27 | Formula 28 | Formula 29 | Formula 30 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Copolymer a2)* | 0.1 to 9.9 | 0.1 to 8.9 | 0.5 to 8.0 | 0.5 to 7.5 | 1.0 to 7.0 |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |

| | Formula 31 | Formula 32 | Formula 33 | Formula 34 | Formula 35 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer | | | | | |
| (INCI) | | | | | |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |

| | Formula 36 | Formula 37 | Formula 38 | Formula 39 | Formula 40 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer (INCI) | | | | | |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |

*according to claim 1

Of course, not only can the proportion by weight of the liquid carrier based on the total weight of the cosmetic preparation a) vary, but the weight ratio of aqueous to alcoholic carrier is also variable.

The composition of some technically advantageous cosmetic preparations a) having a liquid carrier, in which the proportion by weight of the copolymers a1) and a2) based on the total weight of the cosmetic preparation is about 1.0 to about 10% by weight, preferably about 1.5 to about 9.0% by weight and in particular about 2.0 to about 8.0% by weight, can be found in the following tables. (Unless indicated otherwise, figures specified in % by weight relate to the total weight of the cosmetic agent.) Such cosmetic preparations are suitable for example as pump sprays.

Cosmetic preparations a) having a high ethanol content

| | Formula 41 | Formula 42 | Formula 43 | Formula 44 | Formula 45 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Ethanol | 60 to 98 | 60 to 98 | 60 to 98 | 60 to 98 | 60 to 98 |
| Water | 0 to 10 | 0 to 10 | 0 to 10 | 0 to 10 | 0 to 10 |
| Optional additives | ad 100 | ad 100 | ad 100 | ad 100 | ad 100 |

| | Formula 46 | Formula 47 | Formula 48 | Formula 49 | Formula 50 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Copolymer a2)* | 0.1 to 9.9 | 0.1 to 8.9 | 0.5 to 8.0 | 0.5 to 7.5 | 1.0 to 7.0 |
| Ethanol | 60 to 98 | 60 to 98 | 60 to 98 | 60 to 98 | 60 to 98 |
| Water | 0 to 10 | 0 to 10 | 0 to 10 | 0 to 10 | 0 to 10 |
| Optional additives | ad 100 | ad 100 | ad 100 | ad 100 | ad 100 |

| | Formula 51 | Formula 52 | Formula 53 | Formula 54 | Formula 55 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer | | | | | |
| (INCI) | | | | | |
| Ethanol | 60 to 98 | 60 to 98 | 60 to 98 | 60 to 98 | 60 to 98 |
| Water | 0 to 10 | 0 to 10 | 0 to 10 | 0 to 10 | 0 to 10 |
| Optional additives | ad 100 | ad 100 | ad 100 | ad 100 | ad 100 |

| | Formula 56 | Formula 57 | Formula 58 | Formula 59 | Formula 60 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer (INCI) | | | | | |
| Ethanol | 60 to 98 | 60 to 98 | 60 to 98 | 60 to 98 | 60 to 98 |
| Water | 0 to 10 | 0 to 10 | 0 to 10 | 0 to 10 | 0 to 10 |
| Optional additives | ad 100 | ad 100 | ad 100 | ad 100 | ad 100 |

*according to claim 1

Cosmetic preparations a) having a medium ethanol content

| | Formula 61 | Formula 62 | Formula 63 | Formula 64 | Formula 65 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Ethanol | 10 to 50 | 15 to 50 | 20 to 50 | 25 to 50 | 30 to 50 |
| Water | 2.0 to 40 | 2.0 to 40 | 2.0 to 40 | 2.0 to 40 | 2.0 to 40 |
| Optional additives | ad 100 | ad 100 | ad 100 | ad 100 | ad 100 |

| | Formula 66 | Formula 67 | Formula 68 | Formula 69 | Formula 70 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Copolymer a2)* | 0.1 to 9.9 | 0.1 to 8.9 | 0.5 to 8.0 | 0.5 to 7.5 | 1.0 to 7.0 |
| Ethanol | 10 to 50 | 15 to 50 | 20 to 50 | 25 to 50 | 30 to 50 |
| Water | 2.0 to 40 | 2.0 to 40 | 2.0 to 40 | 2.0 to 40 | 2.0 to 40 |
| Optional additives | ad 100 | ad 100 | ad 100 | ad 100 | ad 100 |

| | Formula 71 | Formula 72 | Formula 73 | Formula 74 | Formula 75 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer | | | | | |
| (INCI) | | | | | |
| Ethanol | 10 to 50 | 15 to 50 | 20 to 50 | 25 to 50 | 30 to 50 |
| Water | 2.0 to 40 | 2.0 to 40 | 2.0 to 40 | 2.0 to 40 | 2.0 to 40 |
| Optional additives | ad 100 | ad 100 | ad 100 | ad 100 | ad 100 |

| | Formula 76 | Formula 77 | Formula 78 | Formula 79 | Formula 80 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer (INCI) | | | | | |
| Ethanol | 10 to 50 | 15 to 50 | 20 to 50 | 25 to 50 | 30 to 50 |
| Water | 2.0 to 40 | 2.0 to 40 | 2.0 to 40 | 2.0 to 40 | 2.0 to 40 |
| Optional additives | ad 100 | ad 100 | ad 100 | ad 100 | ad 100 |

*according to claim 1

Cosmetic preparations a) having a low ethanol content

| | Formula 81 | Formula 82 | Formula 83 | Formula 84 | Formula 85 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Ethanol | 0 to 20 | 0 to 20 | 0 to 20 | 0 to 5.0 | 0 to 5.0 |
| Water | 50 to 97 | 60 to 97 | 60 to 97 | 65 to 97 | 65 to 97 |
| Optional additives | ad 100 | ad 100 | ad 100 | ad 100 | ad 100 |

| | Formula 86 | Formula 87 | Formula 88 | Formula 89 | Formula 90 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Copolymer a2)* | 0.1 to 9.9 | 0.1 to 8.9 | 0.5 to 8.0 | 0.5 to 7.5 | 1.0 to 7.0 |
| Ethanol | 0 to 20 | 0 to 20 | 0 to 20 | 0 to 5.0 | 0 to 5.0 |
| Water | 50 to 97 | 60 to 97 | 60 to 97 | 65 to 97 | 65 to 97 |
| Optional additives | ad 100 | ad 100 | ad 100 | ad 100 | ad 100 |

| | Formula 91 | Formula 92 | Formula 93 | Formula 94 | Formula 95 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer | | | | | |
| (INCI) | | | | | |
| Ethanol | 0 to 20 | 0 to 20 | 0 to 20 | 0 to 5.0 | 0 to 5.0 |
| Water | 50 to 97 | 60 to 97 | 60 to 97 | 65 to 97 | 65 to 97 |
| Optional additives | ad 100 | ad 100 | ad 100 | ad 100 | ad 100 |

| | Formula 96 | Formula 97 | Formula 98 | Formula 99 | Formula 100 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer (INCI) | | | | | |
| Ethanol | 0 to 20 | 0 to 20 | 0 to 20 | 0 to 5.0 | 0 to 5.0 |
| Water | 50 to 97 | 60 to 97 | 60 to 97 | 65 to 97 | 65 to 97 |
| Optional additives | ad 100 | ad 100 | ad 100 | ad 100 | ad 100 |

*according to claim 1

Conceivable made-up forms for cosmetic preparations a) as contemplated herein are creams and lotions but also gels. In addition, however, these preparations are also suitable for use as a mousse, foam or spray.

Cosmetic preparations in gel form contain at least one thickener as a further constituent. With regard to the ease of production, ease of application and cosmetic effect of cosmetic compositions as contemplated herein, it has proven to be advantageous if the proportion by weight of the thickener a2) based on the total weight of the cosmetic preparation a) is about 0.05 to about 8.0% by weight, preferably about 0.1 to about 5.0% by weight.

Preferred thickeners are selected from the group of polymeric organic thickeners. The polymeric organic thickeners may be crosslinked or uncrosslinked.

Preferred thickeners are selected from the group of anionic polymeric organic thickeners. A first group of particularly preferred thickeners a2) contain at least one structural unit selected from at least one structural unit of formula (I) or the salt forms thereof or at least one structural unit (II) or the salt forms thereof

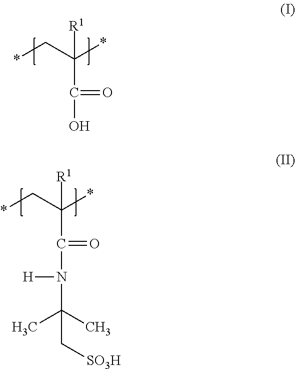

in which R¹ and R² independently of one another represent a hydrogen atom or a methyl group.

According to the formulae above and all the formulae below, a chemical bond denoted by the symbol * represents a free valency of the structural fragment in question.

Particularly preferred anionic polymers which have a thickening effect contain at least one structural unit of formula (I). Acrylic acid homopolymers form a first group of particularly preferred thickeners.

Particularly preferred thickeners are
polyacrylic acids having the INCI name Carbomer, as sold for example by the company 3V Sigma under the trade name Synthalen® K or by the company Lubrizol under the trade name Carbopol.

The anionic, polymeric, amphiphilic thickeners form a second particularly preferred group of thickeners a2). Such thickeners preferably comprise at least one structural unit of formula (III) and at least one structural unit of formula (IV)

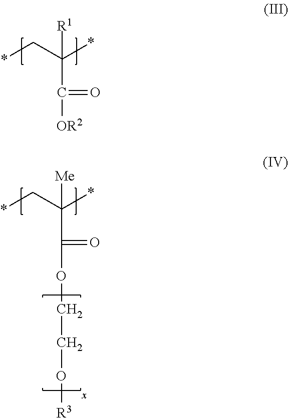

in which

R¹ represents a hydrogen atom or a methyl group,

R² represents a hydrogen atom or a (C₁ to C₆) alkyl group,

R³ represents a (C₈ to C₃₀) alkyl group,

M⁺ represents a physiologically acceptable cation, and

x represents an integer from 0 to 35.

Preference is given in particular to the thickeners having the INCI names Acrylates/Beheneth-25 Methacrylate Copolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-20 Acrylate Copolymer, Acrylates/Palmeth-25 Acrylate Copolymer, Acrylates/Palmeth-25 Itaconate Copolymer and Acrylates/Steareth-50 Acrylate Copolymer.

Particularly preferred thickeners are
thickeners having the INCI name Acrylates/Steareth-20 Methacrylate Copolymer, as sold for example under the trade name Aculyn® 22 by the company Rohm&Haas;
thickeners having the INCI name Acrylates/Steareth-20 Methacrylate Crosspolymer, as sold for example under the trade name Aculyn® 88 by the company Rohm&Haas;
thickeners having the INCI name Acrylates/Steareth-20 Itaconate Copolymer, as sold for example under the trade name Structure 2001 by the company National Starch.

Further anionic, polymeric, amphiphilic thickeners are characterized by long-chain alkyl substituents. This group includes for example the compounds having the INCI names Acrylates/Stearyl Methacrylate Copolymer, Acrylates/Vinyl Isodecanoate Crosspolymer.

Particularly preferred thickeners are
thickeners having the INCI name Acrylates/C₁₀₋₃₀ Alkyl Acrylate Crosspolymer, as sold for example under the trade name Carbopol Ultrez 21 by the company Lubrizol.

Further thickeners may be selected for example from the polymeric thickening agents known under the following INCI names: Acrylamides Copolymer, Acrylamide/Sodium Acrylate Copolymer, Acrylamide/Sodium Acryloyldimethyltaurate Copolymer, Acrylates/Acetoacetoxyethyl Methacrylate Copolymer, Acrylic Acid/Acrylonitrogens Copolymer, Agar, Agarose, Alcaligenes Polysaccharides, Algin, Alginic Acid, Ammonium Acrylates/Acrylonitrogens Copolymer, Ammonium Acrylates Copolymer, Ammonium Acryloyldimethyltaurate/Vinyl Formamide Copolymer, Ammonium Acryloyldimethyltaurate/VP Copolymer, Ammonium Alginate, Ammonium Polyacryloyldimethyl Taurate, Amylopectin, Ascorbyl Methylsilanol Pectinate, Astragalus Gummifer Gum, Attapulgite, Avena Sativa (Oat) Kernel Flour, Bentonite, Butoxy Chitosan, Caesalpinia Spinosa Gum, Calcium Alginate, Calcium Carboxymethyl Cellulose, Calcium Carrageenan, Calcium Potassium Carbomer, Calcium Starch Octenylsuccinate, C20-40 Alkyl Stearate, Carboxybutyl Chitosan, Carboxymethyl Chitin, Carboxymethyl Chitosan, Carboxymethyl Dextran, Carboxymethyl Hydroxyethylcellulose, Carboxymethyl Hydroxypropyl Guar, Cellulose Acetate Propionate Carboxylate, Cellulose Gum, Ceratonia Siliqua Gum, Cetyl Hydroxyethylcellulose, Cholesterol/HDI/Pullulan Copolymer, Cholesteryl Hexyl Dicarbamate Pullulan, Cyamopsis Tetragonoloba (Guar) Gum, Diglycol/CHDM/Isophthalates/SIP Copolymer, Dihydrogenated Tallow Benzylmonium Hectorite, Dimethicone Crosspolymer-2, Dimethicone Propyl PG-Betaine, DMAPA Acrylates/Acrylic Acid/Acrylonitrogens Copolymer, Ethylene/Sodium Acrylate Copolymer, Gelatin, Gellan Gum, Glyceryl Alginate, Glycine Soja (Soybean) Flour, Guar Hydroxypropyltrimonium Chloride, Hectorite, Hydrated Silica, Hydrogenated Potato Starch, Hydroxybutyl Methylcellulose, Hydroxyethyl Acrylate/Sodium Acryloyldimethyl Taurate Copolymer, Hydroxyethylcellulose, Hydroxyethyl Chitosan, Hydroxyethyl Ethylcellulose, Hydroxypropylcellulose, Hydroxypropyl Chitosan, Hydroxypropyl Ethylenediamine Carbomer, Hydroxypropyl Guar, Hydroxypropyl Methylcellulose, Hydroxypropyl Methylcellulose Stearoxy Ether, Hydroxystearamide MEA, Isobutylene/Sodium Maleate Copolymer, Lithium Magnesium Silicate, Lithium Magnesium Sodium Silicate, Macrocystis Pyrifera (Kelp), Magnesium Alginate, Magnesium Aluminum Silicate, Magnesium Silicate, Magnesium Trisilicate, Methoxy PEG-22/Dodecyl Glycol Copolymer, Methylcellulose, Methyl Ethylcellulose, Methyl Hydroxyethylcellulose, Microcrystalline Cellulose, Montmorillonite, Moroccan Lava Clay, Natto Gum, Nonoxynyl Hydroxyethylcellulose, Octadecene/MA Copolymer, Pectin, PEG-800, PEG-Crosspolymer, PEG-150/Decyl Alcohol/SMDI Copolymer, PEG-175 Diisostearate, PEG-190 Distearate, PEG-15 Glyceryl Tristearate, PEG-140 Glyceryl Tristearate, PEG-240/HDI Copolymer Bis-Decyltetradeceth-20 Ether, PEG-100/IPDI Copolymer, PEG-180/Laureth-50/TMMG Copolymer, PEG-10/Lauryl Dimethicone Crosspolymer, PEG-15/Lauryl Dimethicone Crosspolymer, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG-120 Methyl Glucose Trioleate, PEG-180/Octoxynol-40/TMMG Copolymer, PEG-150 Pentaerythrityl Tetrastearate, PEG-4 Rapeseedamide, PEG-150/Stearyl Alcohol/SMDI Copolymer, Polyacrylate-3, Polyacrylic Acid, Polycyclopentadiene, Polyether-1, Polyethylene/Isopropyl Maleate/MA Copolyol, Polymethacrylic Acid, Polyquaternium-52, Polyvinyl Alcohol, Potassium Alginate, Potassium Aluminum Polyacrylate, Potassium Carbomer, Potassium Carrageenan, Potassium Polyacrylate, Potato Starch Modified, PPG-14 Laureth-60 Hexyl Dicarbamate, PPG-14 Laureth-60 Isophoryl Dicarbamate, PPG-14 Palmeth-60 Hexyl Dicarbamate, Propylene Glycol Alginate, PVP/Decene Copolymer, PVP Montmorillonite, Rhizobian Gum, Ricinoleic Acid/Adipic Acid/AEEA Copolymer, Sclerotium Gum, Sodium Acrylate/Acryloyldimethyl Taurate Copolymer, Sodium Acrylates/Acrolein Copolymer, Sodium Acrylates/Acrylonitrogens Copolymer, Sodium Acrylates Copolymer, Sodium Acrylates/Vinyl Isodecanoate Crosspolymer, Sodium Acrylate/Vinyl Alcohol Copolymer, Sodium Carbomer, Sodium Carboxymethyl Chitin, Sodium Carboxymethyl Dextran, Sodium Carboxymethyl Beta-Glucan, Sodium Carboxymethyl Starch, Sodium Carrageenan, Sodium Cellulose Sulfate, Sodium Cyclodextrin Sulfate, Sodium Hydroxypropyl Starch Phosphate, Sodium Isooctylene/MA Copolymer, Sodium Magnesium Fluorosilicate, Sodium Polyacrylate, Sodium Polyacrylate Starch, Sodium Polyacryloyldimethyl Taurate, Sodium Polymethacrylate, Sodium Polystyrene Sulfonate, Sodium Silicoaluminate, Sodium Starch Octenylsuccinate, Sodium Stearoxy PG-Hydroxyethylcellulose Sulfonate, Sodium Styrene/Acrylates Copolymer, Sodium Tauride Acrylates/Acrylic Acid/Acrylonitrogens Copolymer, Solanum Tuberosum (Potato) Starch, Starch/Acrylates/Acrylamide Copolymer, Starch Hydroxypropyltrimonium Chloride, Steareth-60 Cetyl Ether, Steareth-100/PEG-136/HDI Copolymer, Sterculia Urens Gum, Synthetic Fluorphlogopite, Tamarindus Indica Seed Gum, Tapioca Starch, TEA-Alginate, TEA-Carbomer, Triticum Vulgare (Wheat) Starch, Tromethamine Acrylates/Acrylonitrogens Copolymer, Tromethamine Magnesium Aluminum Silicate, Welan Gum, Yeast Beta-Glucan, Yeast Polysaccharides, Zea Mays (Corn) Starch.

The composition of some technically advantageous cosmetic preparations a) having a liquid carrier, in which the proportion by weight of the copolymers a1) and a2) based on the total weight of the cosmetic preparation is about 1.0 to about 10% by weight, preferably about 1.5 to about 9.0% by weight and in particular about 2.0 to about 8.0% by weight, can be found in the following tables. (Unless indicated otherwise, figures specified in % by weight relate to the total weight of the cosmetic agent.)

| | Formula 101 | Formula 102 | Formula 103 | Formula 104 | Formula 105 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Polymeric organic thickener | 0.05 to 8.0 | 0.05 to 8.0 | 0.1 to 5.0 | 0.1 to 5.0 | 0.1 to 5.0 |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |
| | Formula 106 | Formula 107 | Formula 108 | Formula 109 | Formula 110 |
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Copolymer a2)* | 0.1 to 9.9 | 0.1 to 8.9 | 0.5 to 8.0 | 0.5 to 7.5 | 1.0 to 7.0 |
| Polymeric organic thickener | 0.05 to 8.0 | 0.05 to 8.0 | 0.1 to 5.0 | 0.1 to 5.0 | 0.1 to 5.0 |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |

| | Formula 111 | Formula 112 | Formula 113 | Formula 114 | Formula 115 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer | | | | | |
| (INCI) | | | | | |
| Polymeric organic thickener | 0.05 to 8.0 | 0.05 to 8.0 | 0.1 to 5.0 | 0.1 to 5.0 | 0.1 to 5.0 |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |

| | Formula 116 | Formula 117 | Formula 118 | Formula 119 | Formula 120 |
|---|---|---|---|---|---|
| Octylacrylamide/Acrylates/ | 0.1 to 9.9 | 0.1 to 8.9 | 0.5 to 8.0 | 0.5 to 7.5 | 1.0 to 7.0 |
| Butylaminoethyl | | | | | |
| Methacrylate Copolymer | | | | | |
| (INCI) | | | | | |
| Acrylates/Hydroxyesters | 0.1 to 9.9 | 0.1 to 8.9 | 0.5 to 8.0 | 0.5 to 7.5 | 1.0 to 7.0 |
| Acrylates Copolymer (INCI) | | | | | |
| Polymeric organic thickener | 0.05 to 8.0 | 0.05 to 8.0 | 0.1 to 5.0 | 0.1 to 5.0 | 0.1 to 5.0 |
| Water and/or ethanol | 40 to 98 | 40 to 98 | 60 to 95 | 60 to 95 | 70 to 92 |
| Optional additives | ad 100 | ad 100 | ad 100 | ad 100 | ad 100 |

*according to claim 1

Mousse, foam or spray may be provided either without the addition of a propellant, for example by means of a mechanical pumping, foaming or spraying device, or with the use of a propellant (for example aerosol spray). Such cosmetic agents then further comprise at least one propellant b) in addition to the cosmetic preparation a).

Suitable propellants (propellant gases) are propane, propene, n-butane, iso-butane, iso-butene, n-pentane, pentene, iso-pentane, iso-pentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, namely either individually or in combination. Hydrophilic propellant gases, such as for example carbon dioxide, can also advantageously be used within the context of the present disclosure if the proportion of hydrophilic gases is selected to be low and lipophilic propellant gas (for example propane/butane) is present in excess. Particular preference is given to propane, n-butane, iso-butane and mixtures of these propellant gases. Preferred cosmetic agents are characterized in that the agent further comprises at least one propellant b) from the group consisting of propane, mixture of propane and butane, dimethyl ether and 1,1-difluoroethane (INCI: Hydrofluorocarbon 152a).

The further composition of some preferred cosmetic agents which, besides the cosmetic preparation a), also comprise a propellant b), and in which the proportion by weight of the copolymers a1) and a2) based on the total weight of the cosmetic preparation is about 1.0 to about 10% by weight, preferably about 1.5 to about 9.0% by weight and in particular about 2.0 to about 8.0% by weight, can be found in the following tables 1 and 2.

Some preferred cosmetic agents containing cosmetic preparation a) and propellant b) are described in the following tables 1 and 2.

Table 1 shows cosmetic agents having a low propellant content (for example mousses), and table 2 shows cosmetic agents having a high propellant content (for example sprays).

In tables 1 and 2, the left-hand column (“Formula x”) refers to in each case one of the exemplary cosmetic preparations a) of formulae 1 to 100 listed in the tables disclosed above. The further columns two to five (“Propellant”) each indicate the amount of propellant combined with the cosmetic preparation in question. These figures in “% by weight” relate to the total weight of the cosmetic preparation a) of the respective “Formula x” without propellant.

The indication “4 to 12.5% by weight” in the following table 1 therefore corresponds to the addition of propellant to the cosmetic preparation a) in an amount of about 4 to about 12.5% by weight of the weight of the cosmetic preparation a). In other words, in this cosmetic agent, the cosmetic preparation a) and the propellant b) are in a weight ratio of about 100:4 to about 100:12.5 or of about 25:1 to about 8:1.

In other words, the cosmetic agents shown in row 2, column 4 of the following table 1 are mixtures of the propellant-free cosmetic preparation a) according to formula 1 of the above table with propane/butane in a weight ratio of about 100:4 to about 100:12.5 or, expressed differently, a cosmetic agent for the temporary shaping of keratin fibers, comprising
a) a cosmetic preparation, containing
   a1) about 0.1 to about 9.9% by weight of at least one copolymer which is composed at least of the following monomer units:
      N-tert-octylacrylamide,
      acrylic acid,
      tert-butylaminoethyl methacrylate;
   a2) about 0.1 to about 9.9% by weight of at least one copolymer which is composed at least of the following monomer units:
      (meth)acrylic acid,
      (meth)acrylic acid alkyl ester,
      (meth)acrylic acid hydroxyalkyl ester,
b) propellant from the group propane/butane,
   wherein the proportion by weight of the copolymers a1) and a2) based on the total weight of the cosmetic preparation is about 1.0 to about 10% by weight and the weight ratio of cosmetic preparation a) to propellant b) is about 25:1 to about 8:1.

A first group of particularly preferred cosmetic agents as contemplated herein contains, based on its total weight, about 80 to about 96% by weight of the cosmetic preparation a) and about 4 to about 20% by weight propellant, preferably about 87.5 to about 96% by weight of the cosmetic preparation a) and about 4 to about 12.5% by weight propellant b), and in particular about 92 to about 96% by weight of the cosmetic preparation a) and about 4 to about 8% by weight propellant. Preferred propellants are propane/butane mixtures. Such agents are particularly suitable for use as a mousse or foam:

TABLE 1

| | Propellant [% by weight] | | | |
|---|---|---|---|---|
| Formula 1 | 4 to 20 | 4 to 20 P/B* | 4 to 12.5 P/B* | 4 to 8 P/B* |
| Formula 2 | 4 to 20 | 4 to 20 P/B* | 4 to 12.5 P/B* | 4 to 8 P/B* |
| Formula 3 | 4 to 20 | 4 to 20 P/B* | 4 to 12.5 P/B* | 4 to 8 P/B* |
| Formula 4 | 4 to 20 | 4 to 20 P/B* | 4 to 12.5 P/B* | 4 to 8 P/B* |
| Formula 5 | 4 to 20 | 4 to 20 P/B* | 4 to 12.5 P/B* | 4 to 8 P/B* |
| Formula 6 | 4 to 20 | 4 to 20 P/B* | 4 to 12.5 P/B* | 4 to 8 P/B* |
| Formula 7 | 4 to 20 | 4 to 20 P/B* | 4 to 12.5 P/B* | 4 to 8 P/B* |
| Formula 8 | 4 to 20 | 4 to 20 P/B* | 4 to 12.5 P/B* | 4 to 8 P/B* |
| Formula 9 | 4 to 20 | 4 to 20 P/B* | 4 to 12.5 P/B* | 4 to 8 P/B* |
| Formula 10 | 4 to 20 | 4 to 20 P/B* | 4 to 12.5 P/B* | 4 to 8 P/B* |
| Formula 11 | 4 to 20 | 4 to 20 P/B* | 4 to 12.5 P/B* | 4 to 8 P/B* |
| Formula 12 | 4 to 20 | 4 to 20 P/B* | 4 to 12.5 P/B* | 4 to 8 P/B* |
| Formula 13 | 4 to 20 | 4 to 20 P/B* | 4 to 12.5 P/B* | 4 to 8 P/B* |
| Formula 14 | 4 to 20 | 4 to 20 P/B* | 4 to 12.5 P/B* | 4 to 8 P/B* |
| Formula 15 | 4 to 20 | 4 to 20 P/B* | 4 to 12.5 P/B* | 4 to 8 P/B* |
| Formula 16 | 4 to 20 | 4 to 20 P/B* | 4 to 12.5 P/B* | 4 to 8 P/B* |
| Formula 17 | 4 to 20 | 4 to 20 P/B* | 4 to 12.5 P/B* | 4 to 8 P/B* |
| Formula 18 | 4 to 20 | 4 to 20 P/B* | 4 to 12.5 P/B* | 4 to 8 P/B* |
| Formula 19 | 4 to 20 | 4 to 20 P/B* | 4 to 12.5 P/B* | 4 to 8 P/B* |
| Formula 20 | 4 to 20 | 4 to 20 P/B* | 4 to 12.5 P/B* | 4 to 8 P/B* |
| Formula 21 | 4 to 20 | 4 to 20 P/B* | 4 to 12.5 P/B* | 4 to 8 P/B* |
| Formula 22 | 4 to 20 | 4 to 20 P/B* | 4 to 12.5 P/B* | 4 to 8 P/B* |
| Formula 23 | 4 to 20 | 4 to 20 P/B* | 4 to 12.5 P/B* | 4 to 8 P/B* |
| Formula 24 | 4 to 20 | 4 to 20 P/B* | 4 to 12.5 P/B* | 4 to 8 P/B* |
| Formula 25 | 4 to 20 | 4 to 20 P/B* | 4 to 12.5 P/B* | 4 to 8 P/B* |
| Formula 26 | 4 to 20 | 4 to 20 P/B* | 4 to 12.5 P/B* | 4 to 8 P/B* |
| Formula 27 | 4 to 20 | 4 to 20 P/B* | 4 to 12.5 P/B* | 4 to 8 P/B* |
| Formula 28 | 4 to 20 | 4 to 20 P/B* | 4 to 12.5 P/B* | 4 to 8 P/B* |
| Formula 29 | 4 to 20 | 4 to 20 P/B* | 4 to 12.5 P/B* | 4 to 8 P/B* |
| Formula 30 | 4 to 20 | 4 to 20 P/B* | 4 to 12.5 P/B* | 4 to 8 P/B* |
| Formula 31 | 4 to 20 | 4 to 20 P/B* | 4 to 12.5 P/B* | 4 to 8 P/B* |
| Formula 32 | 4 to 20 | 4 to 20 P/B* | 4 to 12.5 P/B* | 4 to 8 P/B* |
| Formula 33 | 4 to 20 | 4 to 20 P/B* | 4 to 12.5 P/B* | 4 to 8 P/B* |
| Formula 34 | 4 to 20 | 4 to 20 P/B* | 4 to 12.5 P/B* | 4 to 8 P/B* |
| Formula 35 | 4 to 20 | 4 to 20 P/B* | 4 to 12.5 P/B* | 4 to 8 P/B* |
| Formula 36 | 4 to 20 | 4 to 20 P/B* | 4 to 12.5 P/B* | 4 to 8 P/B* |
| Formula 37 | 4 to 20 | 4 to 20 P/B* | 4 to 12.5 P/B* | 4 to 8 P/B* |
| Formula 38 | 4 to 20 | 4 to 20 P/B* | 4 to 12.5 P/B* | 4 to 8 P/B* |
| Formula 39 | 4 to 20 | 4 to 20 P/B* | 4 to 12.5 P/B* | 4 to 8 P/B* |
| Formula 40 | 4 to 20 | 4 to 20 P/B* | 4 to 12.5 P/B* | 4 to 8 P/B* |
| Formula 41 | 4 to 20 | 4 to 20 P/B* | 4 to 12.5 P/B* | 4 to 8 P/B* |
| Formula 42 | 4 to 20 | 4 to 20 P/B* | 4 to 12.5 P/B* | 4 to 8 P/B* |
| Formula 43 | 4 to 20 | 4 to 20 P/B* | 4 to 12.5 P/B* | 4 to 8 P/B* |
| Formula 44 | 4 to 20 | 4 to 20 P/B* | 4 to 12.5 P/B* | 4 to 8 P/B* |
| Formula 45 | 4 to 20 | 4 to 20 P/B* | 4 to 12.5 P/B* | 4 to 8 P/B* |
| Formula 46 | 4 to 20 | 4 to 20 P/B* | 4 to 12.5 P/B* | 4 to 8 P/B* |
| Formula 47 | 4 to 20 | 4 to 20 P/B* | 4 to 12.5 P/B* | 4 to 8 P/B* |
| Formula 48 | 4 to 20 | 4 to 20 P/B* | 4 to 12.5 P/B* | 4 to 8 P/B* |
| Formula 49 | 4 to 20 | 4 to 20 P/B* | 4 to 12.5 P/B* | 4 to 8 P/B* |
| Formula 50 | 4 to 20 | 4 to 20 P/B* | 4 to 12.5 P/B* | 4 to 8 P/B* |
| Formula 51 | 4 to 20 | 4 to 20 P/B* | 4 to 12.5 P/B* | 4 to 8 P/B* |
| Formula 52 | 4 to 20 | 4 to 20 P/B* | 4 to 12.5 P/B* | 4 to 8 P/B* |
| Formula 53 | 4 to 20 | 4 to 20 P/B* | 4 to 12.5 P/B* | 4 to 8 P/B* |
| Formula 54 | 4 to 20 | 4 to 20 P/B* | 4 to 12.5 P/B* | 4 to 8 P/B* |
| Formula 55 | 4 to 20 | 4 to 20 P/B* | 4 to 12.5 P/B* | 4 to 8 P/B* |
| Formula 56 | 4 to 20 | 4 to 20 P/B* | 4 to 12.5 P/B* | 4 to 8 P/B* |
| Formula 57 | 4 to 20 | 4 to 20 P/B* | 4 to 12.5 P/B* | 4 to 8 P/B* |
| Formula 58 | 4 to 20 | 4 to 20 P/B* | 4 to 12.5 P/B* | 4 to 8 P/B* |
| Formula 59 | 4 to 20 | 4 to 20 P/B* | 4 to 12.5 P/B* | 4 to 8 P/B* |
| Formula 60 | 4 to 20 | 4 to 20 P/B* | 4 to 12.5 P/B* | 4 to 8 P/B* |
| Formula 61 | 4 to 20 | 4 to 20 P/B* | 4 to 12.5 P/B* | 4 to 8 P/B* |
| Formula 62 | 4 to 20 | 4 to 20 P/B* | 4 to 12.5 P/B* | 4 to 8 P/B* |
| Formula 63 | 4 to 20 | 4 to 20 P/B* | 4 to 12.5 P/B* | 4 to 8 P/B* |
| Formula 64 | 4 to 20 | 4 to 20 P/B* | 4 to 12.5 P/B* | 4 to 8 P/B* |
| Formula 65 | 4 to 20 | 4 to 20 P/B* | 4 to 12.5 P/B* | 4 to 8 P/B* |
| Formula 66 | 4 to 20 | 4 to 20 P/B* | 4 to 12.5 P/B* | 4 to 8 P/B* |
| Formula 67 | 4 to 20 | 4 to 20 P/B* | 4 to 12.5 P/B* | 4 to 8 P/B* |
| Formula 68 | 4 to 20 | 4 to 20 P/B* | 4 to 12.5 P/B* | 4 to 8 P/B* |
| Formula 69 | 4 to 20 | 4 to 20 P/B* | 4 to 12.5 P/B* | 4 to 8 P/B* |
| Formula 70 | 4 to 20 | 4 to 20 P/B* | 4 to 12.5 P/B* | 4 to 8 P/B* |
| Formula 71 | 4 to 20 | 4 to 20 P/B* | 4 to 12.5 P/B* | 4 to 8 P/B* |
| Formula 72 | 4 to 20 | 4 to 20 P/B* | 4 to 12.5 P/B* | 4 to 8 P/B* |
| Formula 73 | 4 to 20 | 4 to 20 P/B* | 4 to 12.5 P/B* | 4 to 8 P/B* |
| Formula 74 | 4 to 20 | 4 to 20 P/B* | 4 to 12.5 P/B* | 4 to 8 P/B* |
| Formula 75 | 4 to 20 | 4 to 20 P/B* | 4 to 12.5 P/B* | 4 to 8 P/B* |
| Formula 76 | 4 to 20 | 4 to 20 P/B* | 4 to 12.5 P/B* | 4 to 8 P/B* |
| Formula 77 | 4 to 20 | 4 to 20 P/B* | 4 to 12.5 P/B* | 4 to 8 P/B* |
| Formula 78 | 4 to 20 | 4 to 20 P/B* | 4 to 12.5 P/B* | 4 to 8 P/B* |
| Formula 79 | 4 to 20 | 4 to 20 P/B* | 4 to 12.5 P/B* | 4 to 8 P/B* |
| Formula 80 | 4 to 20 | 4 to 20 P/B* | 4 to 12.5 P/B* | 4 to 8 P/B* |
| Formula 81 | 4 to 20 | 4 to 20 P/B* | 4 to 12.5 P/B* | 4 to 8 P/B* |
| Formula 82 | 4 to 20 | 4 to 20 P/B* | 4 to 12.5 P/B* | 4 to 8 P/B* |
| Formula 83 | 4 to 20 | 4 to 20 P/B* | 4 to 12.5 P/B* | 4 to 8 P/B* |
| Formula 84 | 4 to 20 | 4 to 20 P/B* | 4 to 12.5 P/B* | 4 to 8 P/B* |
| Formula 85 | 4 to 20 | 4 to 20 P/B* | 4 to 12.5 P/B* | 4 to 8 P/B* |
| Formula 86 | 4 to 20 | 4 to 20 P/B* | 4 to 12.5 P/B* | 4 to 8 P/B* |
| Formula 87 | 4 to 20 | 4 to 20 P/B* | 4 to 12.5 P/B* | 4 to 8 P/B* |
| Formula 88 | 4 to 20 | 4 to 20 P/B* | 4 to 12.5 P/B* | 4 to 8 P/B* |
| Formula 89 | 4 to 20 | 4 to 20 P/B* | 4 to 12.5 P/B* | 4 to 8 P/B* |
| Formula 90 | 4 to 20 | 4 to 20 P/B* | 4 to 12.5 P/B* | 4 to 8 P/B* |
| Formula 91 | 4 to 20 | 4 to 20 P/B* | 4 to 12.5 P/B* | 4 to 8 P/B* |
| Formula 92 | 4 to 20 | 4 to 20 P/B* | 4 to 12.5 P/B* | 4 to 8 P/B* |
| Formula 93 | 4 to 20 | 4 to 20 P/B* | 4 to 12.5 P/B* | 4 to 8 P/B* |
| Formula 94 | 4 to 20 | 4 to 20 P/B* | 4 to 12.5 P/B* | 4 to 8 P/B* |
| Formula 95 | 4 to 20 | 4 to 20 P/B* | 4 to 12.5 P/B* | 4 to 8 P/B* |
| Formula 96 | 4 to 20 | 4 to 20 P/B* | 4 to 12.5 P/B* | 4 to 8 P/B* |
| Formula 97 | 4 to 20 | 4 to 20 P/B* | 4 to 12.5 P/B* | 4 to 8 P/B* |
| Formula 98 | 4 to 20 | 4 to 20 P/B* | 4 to 12.5 P/B* | 4 to 8 P/B* |
| Formula 99 | 4 to 20 | 4 to 20 P/B* | 4 to 12.5 P/B* | 4 to 8 P/B* |
| Formula 100 | 4 to 20 | 4 to 20 P/B* | 4 to 12.5 P/B* | 4 to 8 P/B* |

*“P/B” denotes a propane/butane mixture

The indication “50 to 200% by weight” in the following table 2 corresponds to the addition of propellant to the cosmetic preparation a) in an amount of about 50 to about 200% by weight of the weight of the cosmetic preparation a). In other words, in this cosmetic agent, the cosmetic preparation a) and the propellant b) are in a weight ratio of about 100:50 to about 100:200 or of about 2:1 to about 1:2.

Row 4, column 3 of the following table 2 therefore describes a mixture of the propellant-free cosmetic preparation a) according to formula 3 with a propane/butane mixture. The entry in row 4, column 3 therefore describes a cosmetic agent for the temporary shaping of keratin fibers, comprising
a) a cosmetic preparation, containing
   a1) about 0.5 to about 8.0% by weight of at least one copolymer which is composed at least of the following monomer units:
      N-tert-octylacrylamide,
      acrylic acid,
      tert-butylaminoethyl methacrylate;
   a2) about 0.5 to about 8.0% by weight of at least one copolymer which is composed at least of the following monomer units:
      (meth)acrylic acid,
      (meth)acrylic acid alkyl ester,
      (meth)acrylic acid hydroxyalkyl ester,
b) propellant from the group consisting of propane/butane mixtures,
   wherein the proportion by weight of the copolymers a1) and a2) based on the total weight of the cosmetic preparation is about 1.0 to about 10% by weight and the weight ratio of cosmetic preparation a) to propellant b) is about 2:1 to about 1:2.

A second group of particularly preferred cosmetic agents as contemplated herein contains, based on its total weight, about 30 to about 70% by weight of the cosmetic preparation a) and about 30 to about 70% by weight propellant b). Such agents are particularly suitable for use as a spray:

TABLE 2

| | Propellant [% by weight] | | | |
|---|---|---|---|---|
| Formula 1 | 50 to 200 | 50 to 200 P/B* | 50 to 200 DFE** | 50 to 200 DME*** |
| Formula 2 | 50 to 200 | 50 to 200 P/B* | 50 to 200 DFE** | 50 to 200 DME*** |
| Formula 3 | 50 to 200 | 50 to 200 P/B* | 50 to 200 DFE** | 50 to 200 DME*** |
| Formula 4 | 50 to 200 | 50 to 200 P/B* | 50 to 200 DFE** | 50 to 200 DME*** |
| Formula 5 | 50 to 200 | 50 to 200 P/B* | 50 to 200 DFE** | 50 to 200 DME*** |
| Formula 6 | 50 to 200 | 50 to 200 P/B* | 50 to 200 DFE** | 50 to 200 DME*** |
| Formula 7 | 50 to 200 | 50 to 200 P/B* | 50 to 200 DFE** | 50 to 200 DME*** |
| Formula 8 | 50 to 200 | 50 to 200 P/B* | 50 to 200 DFE** | 50 to 200 DME*** |
| Formula 9 | 50 to 200 | 50 to 200 P/B* | 50 to 200 DFE** | 50 to 200 DME*** |
| Formula 10 | 50 to 200 | 50 to 200 P/B* | 50 to 200 DFE** | 50 to 200 DME*** |
| Formula 11 | 50 to 200 | 50 to 200 P/B* | 50 to 200 DFE** | 50 to 200 DME*** |
| Formula 12 | 50 to 200 | 50 to 200 P/B* | 50 to 200 DFE** | 50 to 200 DME*** |
| Formula 13 | 50 to 200 | 50 to 200 P/B* | 50 to 200 DFE** | 50 to 200 DME*** |
| Formula 14 | 50 to 200 | 50 to 200 P/B* | 50 to 200 DFE** | 50 to 200 DME*** |
| Formula 15 | 50 to 200 | 50 to 200 P/B* | 50 to 200 DFE** | 50 to 200 DME*** |
| Formula 16 | 50 to 200 | 50 to 200 P/B* | 50 to 200 DFE** | 50 to 200 DME*** |
| Formula 17 | 50 to 200 | 50 to 200 P/B* | 50 to 200 DFE** | 50 to 200 DME*** |
| Formula 18 | 50 to 200 | 50 to 200 P/B* | 50 to 200 DFE** | 50 to 200 DME*** |
| Formula 19 | 50 to 200 | 50 to 200 P/B* | 50 to 200 DFE** | 50 to 200 DME*** |
| Formula 20 | 50 to 200 | 50 to 200 P/B* | 50 to 200 DFE** | 50 to 200 DME*** |
| Formula 21 | 50 to 200 | 50 to 200 P/B* | 50 to 200 DFE** | 50 to 200 DME*** |
| Formula 22 | 50 to 200 | 50 to 200 P/B* | 50 to 200 DFE** | 50 to 200 DME*** |
| Formula 23 | 50 to 200 | 50 to 200 P/B* | 50 to 200 DFE** | 50 to 200 DME*** |
| Formula 24 | 50 to 200 | 50 to 200 P/B* | 50 to 200 DFE** | 50 to 200 DME*** |
| Formula 25 | 50 to 200 | 50 to 200 P/B* | 50 to 200 DFE** | 50 to 200 DME*** |
| Formula 26 | 50 to 200 | 50 to 200 P/B* | 50 to 200 DFE** | 50 to 200 DME*** |
| Formula 27 | 50 to 200 | 50 to 200 P/B* | 50 to 200 DFE** | 50 to 200 DME*** |
| Formula 28 | 50 to 200 | 50 to 200 P/B* | 50 to 200 DFE** | 50 to 200 DME*** |
| Formula 29 | 50 to 200 | 50 to 200 P/B* | 50 to 200 DFE** | 50 to 200 DME*** |
| Formula 30 | 50 to 200 | 50 to 200 P/B* | 50 to 200 DFE** | 50 to 200 DME*** |
| Formula 31 | 50 to 200 | 50 to 200 P/B* | 50 to 200 DFE** | 50 to 200 DME*** |
| Formula 32 | 50 to 200 | 50 to 200 P/B* | 50 to 200 DFE** | 50 to 200 DME*** |
| Formula 33 | 50 to 200 | 50 to 200 P/B* | 50 to 200 DFE** | 50 to 200 DME*** |
| Formula 34 | 50 to 200 | 50 to 200 P/B* | 50 to 200 DFE** | 50 to 200 DME*** |
| Formula 35 | 50 to 200 | 50 to 200 P/B* | 50 to 200 DFE** | 50 to 200 DME*** |
| Formula 36 | 50 to 200 | 50 to 200 P/B* | 50 to 200 DFE** | 50 to 200 DME*** |
| Formula 37 | 50 to 200 | 50 to 200 P/B* | 50 to 200 DFE** | 50 to 200 DME*** |
| Formula 38 | 50 to 200 | 50 to 200 P/B* | 50 to 200 DFE** | 50 to 200 DME*** |
| Formula 39 | 50 to 200 | 50 to 200 P/B* | 50 to 200 DFE** | 50 to 200 DME*** |
| Formula 40 | 50 to 200 | 50 to 200 P/B* | 50 to 200 DFE** | 50 to 200 DME*** |
| Formula 41 | 50 to 200 | 50 to 200 P/B* | 50 to 200 DFE** | 50 to 200 DME*** |
| Formula 42 | 50 to 200 | 50 to 200 P/B* | 50 to 200 DFE** | 50 to 200 DME*** |
| Formula 43 | 50 to 200 | 50 to 200 P/B* | 50 to 200 DFE** | 50 to 200 DME*** |
| Formula 44 | 50 to 200 | 50 to 200 P/B* | 50 to 200 DFE** | 50 to 200 DME*** |
| Formula 45 | 50 to 200 | 50 to 200 P/B* | 50 to 200 DFE** | 50 to 200 DME*** |
| Formula 46 | 50 to 200 | 50 to 200 P/B* | 50 to 200 DFE** | 50 to 200 DME*** |
| Formula 47 | 50 to 200 | 50 to 200 P/B* | 50 to 200 DFE** | 50 to 200 DME*** |
| Formula 48 | 50 to 200 | 50 to 200 P/B* | 50 to 200 DFE** | 50 to 200 DME*** |
| Formula 49 | 50 to 200 | 50 to 200 P/B* | 50 to 200 DFE** | 50 to 200 DME*** |
| Formula 50 | 50 to 200 | 50 to 200 P/B* | 50 to 200 DFE** | 50 to 200 DME*** |
| Formula 51 | 50 to 200 | 50 to 200 P/B* | 50 to 200 DFE** | 50 to 200 DME*** |
| Formula 52 | 50 to 200 | 50 to 200 P/B* | 50 to 200 DFE** | 50 to 200 DME*** |
| Formula 53 | 50 to 200 | 50 to 200 P/B* | 50 to 200 DFE** | 50 to 200 DME*** |
| Formula 54 | 50 to 200 | 50 to 200 P/B* | 50 to 200 DFE** | 50 to 200 DME*** |
| Formula 55 | 50 to 200 | 50 to 200 P/B* | 50 to 200 DFE** | 50 to 200 DME*** |
| Formula 56 | 50 to 200 | 50 to 200 P/B* | 50 to 200 DFE** | 50 to 200 DME*** |
| Formula 57 | 50 to 200 | 50 to 200 P/B* | 50 to 200 DFE** | 50 to 200 DME*** |
| Formula 58 | 50 to 200 | 50 to 200 P/B* | 50 to 200 DFE** | 50 to 200 DME*** |
| Formula 59 | 50 to 200 | 50 to 200 P/B* | 50 to 200 DFE** | 50 to 200 DME*** |
| Formula 60 | 50 to 200 | 50 to 200 P/B* | 50 to 200 DFE** | 50 to 200 DME*** |
| Formula 61 | 50 to 200 | 50 to 200 P/B* | 50 to 200 DFE** | 50 to 200 DME*** |
| Formula 62 | 50 to 200 | 50 to 200 P/B* | 50 to 200 DFE** | 50 to 200 DME*** |
| Formula 63 | 50 to 200 | 50 to 200 P/B* | 50 to 200 DFE** | 50 to 200 DME*** |
| Formula 64 | 50 to 200 | 50 to 200 P/B* | 50 to 200 DFE** | 50 to 200 DME*** |
| Formula 65 | 50 to 200 | 50 to 200 P/B* | 50 to 200 DFE** | 50 to 200 DME*** |
| Formula 66 | 50 to 200 | 50 to 200 P/B* | 50 to 200 DFE** | 50 to 200 DME*** |
| Formula 67 | 50 to 200 | 50 to 200 P/B* | 50 to 200 DFE** | 50 to 200 DME*** |
| Formula 68 | 50 to 200 | 50 to 200 P/B* | 50 to 200 DFE** | 50 to 200 DME*** |
| Formula 69 | 50 to 200 | 50 to 200 P/B* | 50 to 200 DFE** | 50 to 200 DME*** |
| Formula 70 | 50 to 200 | 50 to 200 P/B* | 50 to 200 DFE** | 50 to 200 DME*** |
| Formula 71 | 50 to 200 | 50 to 200 P/B* | 50 to 200 DFE** | 50 to 200 DME*** |
| Formula 72 | 50 to 200 | 50 to 200 P/B* | 50 to 200 DFE** | 50 to 200 DME*** |
| Formula 73 | 50 to 200 | 50 to 200 P/B* | 50 to 200 DFE** | 50 to 200 DME*** |
| Formula 74 | 50 to 200 | 50 to 200 P/B* | 50 to 200 DFE** | 50 to 200 DME*** |
| Formula 75 | 50 to 200 | 50 to 200 P/B* | 50 to 200 DFE** | 50 to 200 DME*** |
| Formula 76 | 50 to 200 | 50 to 200 P/B* | 50 to 200 DFE** | 50 to 200 DME*** |
| Formula 77 | 50 to 200 | 50 to 200 P/B* | 50 to 200 DFE** | 50 to 200 DME*** |
| Formula 78 | 50 to 200 | 50 to 200 P/B* | 50 to 200 DFE** | 50 to 200 DME*** |
| Formula 79 | 50 to 200 | 50 to 200 P/B* | 50 to 200 DFE** | 50 to 200 DME*** |
| Formula 80 | 50 to 200 | 50 to 200 P/B* | 50 to 200 DFE** | 50 to 200 DME*** |
| Formula 81 | 50 to 200 | 50 to 200 P/B* | 50 to 200 DFE** | 50 to 200 DME*** |
| Formula 82 | 50 to 200 | 50 to 200 P/B* | 50 to 200 DFE** | 50 to 200 DME*** |
| Formula 83 | 50 to 200 | 50 to 200 P/B* | 50 to 200 DFE** | 50 to 200 DME*** |
| Formula 84 | 50 to 200 | 50 to 200 P/B* | 50 to 200 DFE** | 50 to 200 DME*** |
| Formula 85 | 50 to 200 | 50 to 200 P/B* | 50 to 200 DFE** | 50 to 200 DME*** |
| Formula 86 | 50 to 200 | 50 to 200 P/B* | 50 to 200 DFE** | 50 to 200 DME*** |
| Formula 87 | 50 to 200 | 50 to 200 P/B* | 50 to 200 DFE** | 50 to 200 DME*** |
| Formula 88 | 50 to 200 | 50 to 200 P/B* | 50 to 200 DFE** | 50 to 200 DME*** |
| Formula 89 | 50 to 200 | 50 to 200 P/B* | 50 to 200 DFE** | 50 to 200 DME*** |
| Formula 90 | 50 to 200 | 50 to 200 P/B* | 50 to 200 DFE** | 50 to 200 DME*** |
| Formula 91 | 50 to 200 | 50 to 200 P/B* | 50 to 200 DFE** | 50 to 200 DME*** |
| Formula 92 | 50 to 200 | 50 to 200 P/B* | 50 to 200 DFE** | 50 to 200 DME*** |
| Formula 93 | 50 to 200 | 50 to 200 P/B* | 50 to 200 DFE** | 50 to 200 DME*** |
| Formula 94 | 50 to 200 | 50 to 200 P/B* | 50 to 200 DFE** | 50 to 200 DME*** |
| Formula 95 | 50 to 200 | 50 to 200 P/B* | 50 to 200 DFE** | 50 to 200 DME*** |
| Formula 96 | 50 to 200 | 50 to 200 P/B* | 50 to 200 DFE** | 50 to 200 DME*** |
| Formula 97 | 50 to 200 | 50 to 200 P/B* | 50 to 200 DFE** | 50 to 200 DME*** |
| Formula 98 | 50 to 200 | 50 to 200 P/B* | 50 to 200 DFE** | 50 to 200 DME*** |
| Formula 99 | 50 to 200 | 50 to 200 P/B* | 50 to 200 DFE** | 50 to 200 DME*** |
| Formula 100 | 50 to 200 | 50 to 200 P/B* | 50 to 200 DFE** | 50 to 200 DME*** |

*“P/B” denotes a propane/butane mixture
**“DFE” denotes 1,1-difluoroethane
***“DME” denotes dimethyl ether

Suitable pressurized gas containers for aerosol applications are vessels made of metal (aluminum, tinplate, tin), protected or shatterproof plastic, or glass coated with plastic on the outside, wherein the compressive and fracture strength, corrosion resistance, ability to be filled easily and also esthetic aspects, ease of handling, printability, etc. play a role when making a choice. Special internal protective coatings ensure corrosion resistance against the cosmetic agents a).

If the agents as contemplated herein are intended to be sprayed onto the hair, these agents are advantageously provided with a dispensing device and a spray valve. The resulting cosmetic products therefore comprise a cosmetic agent as contemplated herein and a dispensing device with a spray valve.

In one preferred embodiment of the disclosure, the valve has a valve cone coated with a lacquer or a polymer plastic A, and a flexible element with return characteristics which returns the valve to the closed position (=rest position of the valve) at the end of actuation. Such cosmetic products, in which the aerosol dispensing device comprises a valve which has a valve cone and/or a flexible element with return characteristics which is/are coated with a lacquer or a polymeric plastic A, are preferred as contemplated herein.

In a further preferred embodiment of the disclosure, the valve has a flexible element with return characteristics and/or a valve cone made of at least one plastic B, preferably an elastomeric plastic. Here, too, preference is given to cosmetic products as contemplated herein in which the valve has a flexible element with return characteristics and/or a valve cone made of at least one plastic B, wherein preferred plastics B are elastomeric plastics. Particularly preferred elastomeric plastics are selected from Buna, in particular Buna N, Buna 421, Buna 1602 and Buna KA 6712, neoprene, butyl and chlorobutyl.

In a further preferred embodiment of the disclosure, the flexible element with return characteristics may be configured as a coil spring or helical compression spring. In a further preferred embodiment of the disclosure, the flexible element with return characteristics may be formed in one piece with the valve cone and may have flexible legs.

As mentioned in the introduction, the cosmetic agents described above are characterized by particular hair-cosmetic properties, in particular advantageous properties for the temporary shaping of hair. A second subject matter of the present application is therefore the use of an agent as contemplated herein for the temporary shaping of keratin fibers, in particular human hair.

A third subject matter of the present application is a method for the temporary shaping of keratin fibers, in particular human hair, in which a cosmetic agent as contemplated herein is applied to the keratin fibers and the keratin fibers are temporarily fixed in shape.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A cosmetic agent for the temporary shaping of keratin fibers, comprising:
a cosmetic preparation, containing:
at least one copolymer a1) which is composed at least of the following monomer units;
N-tert-octylacrylamide,
acrylic acid, and
tert-butylaminoethyl methacrylate; and
at least one copolymer a2) which is composed at least of the following monomer units;
(meth)acrylic acid,
(meth)acrylic acid alkyl ester, and
(meth)acrylic acid hydroxyalkyl ester;
wherein the proportion by weight of the copolymers a1) and a2) based on the total weight of the cosmetic preparation is about 1.0 to about 10% by weight.

2. The cosmetic agent according to claim 1, wherein the proportion by weight of the copolymers a1) and a2) based on the total weight of the cosmetic preparation is about 1.5 to about 9.0% by weight.

3. The cosmetic agent according to claim 1, wherein the preparation contains, based on its total weight, about 0.1 to about 9.9% by weight of copolymer a1).

4. The cosmetic agent according to claim 1, wherein the preparation contains, based on its total weight, about 0.1 to about 9.9% by weight of copolymer a2).

5. The cosmetic agent according to claim 1, wherein the weight ratio of copolymer a1) to copolymer a2) is about 1:7 to about 7:1.

6. The cosmetic agent according to claim 1, wherein the agent further comprises at least one propellant.

7. The cosmetic agent according to claim 1, comprising, based on its total weight,
a) about 30 to about 70% by weight of the cosmetic preparation; and
b) about 30 to about 70% by weight propellant.

8. A cosmetic product, comprising:
a cosmetic agent according to claim 1; and
a dispensing device with a spray valve.

9. (canceled)

10. (canceled)

11. The cosmetic agent according to claim 1, wherein the proportion by weight of the copolymers a1) and a2) based on the total weight of the cosmetic preparation is about 2.0 to about 8.0% by weight.

12. The cosmetic agent according to claim 1, wherein the preparation contains, based on its total weight, about 0.5 to about 8.5% by weight of copolymer a1).

13. The cosmetic agent according to claim 1, wherein the preparation contains, based on its total weight, about 1.0 to about 7.0% by weight of copolymer a1).

14. The cosmetic agent according to claim 1, wherein the preparation contains, based on its total weight, about 0.5 to about 8.5% by weight of copolymer a2).

15. The cosmetic agent according to claim 1, wherein the preparation contains, based on its total weight, about 1.0 to about 7.0% by weight of copolymer a2).

16. The cosmetic agent according to claim 1, wherein the weight ratio of copolymer a1) to copolymer a2) is about 1:5 to about 5:1.

17. The cosmetic agent according to claim 1, wherein the weight ratio of copolymer a1) to copolymer a2) is about 1:3 to about 3:1.

18. The cosmetic agent according to claim 1, wherein the copolymer a1) consists of the monomers N-tert-octylacrylamide, acrylic acid, tert-butylaminoethyl methacrylate, methyl methacrylate and hydroxypropyl methacrylate in a proportion of at least 90% by weight.

19. The cosmetic agent according to claim 1, wherein the copolymer a1) bears the INCI name Octylacrylamide/Acrylates/Butylaminoethyl Methacrylate Copolymer.

20. The cosmetic agent according to claim 1, wherein the copolymer a2) bears the INCI name Acrylates/Hydroxyesters Acrylates Copolymer.

21. A cosmetic agent for the temporary shaping of keratin fibers, comprising:
a cosmetic preparation, containing:
at least one copolymer a1) which is composed at least of the following monomer units;
N-tert-octylacrylamide,
acrylic acid, and
tert-butylaminoethyl methacrylate;
at least one copolymer a2) which is composed at least of the following monomer units;
(meth)acrylic acid,
(meth)acrylic acid alkyl ester, and
(meth)acrylic acid hydroxyalkyl ester;
at least one propellant from the group propane, mixture of propane and butane, dimethyl ether and 1,1-difluoroethane;
ethanol; and
water;
wherein the proportion by weight of the copolymers a1) and a2) based on the total weight of the cosmetic preparation is about 1.0 to about 10% by weight.

22. A method for the temporary shaping of keratin fibers, the method comprising the steps of:
applying a cosmetic agent to the keratin fibers, the cosmetic agent comprising:
at least one copolymer a1) which is composed at least of the following monomer units;
N-tert-octylacrylamide,
acrylic acid, and
tert-butylaminoethyl methacrylate; and
at least one copolymer a2) which is composed at least of the following monomer units;
(meth)acrylic acid,
(meth)acrylic acid alkyl ester, and
(meth)acrylic acid hydroxyalkyl ester;
wherein the proportion by weight of the copolymers a1) and a2) based on the total weight of the cosmetic agent is about 1.0 to about 10% by weight; and
temporarily fixing the keratin fibers in shape.
